(19) United States
(12) Patent Application Publication
(10) Pub. No.: US 2026/0200537 A1
(43) Pub. Date: Jul. 16, 2026

(54) LIGHTWEIGHT MATERIAL CARRIER

(71) Applicant: ANGLO AMERICAN TECHNICAL & SUSTAINABILITY SERVICES LTD, London (GB)

(72) Inventors: Luke Michael SMITH, London (GB); Daniel Clark FINFER, London (GB)

(21) Appl. No.: 19/138,586

(22) PCT Filed: Dec. 13, 2023
(86) PCT No.: PCT/IB2023/062600

§ 371 Date: Jun. 12, 2025

(30) Foreign Application Priority Data
Dec. 14, 2022 (GB) 2218856.9

(51) Int. Cl. B62D 33/02; E21F 13/02; G01N 33/24

(52) U.S. Cl. CPC B62D 33/02; E21F 13/025; G01N 33/24

(57) ABSTRACT

A lightweight haul vehicle (10) comprising a haul carrier arrangement (16), typically utilised in mining and/or construction operations for hauling raw material. The vehicle (10) comprises a chassis (14) for supporting the haul carrier arrangement (16). The haul carrier arrangement (16) comprises a support arrangement (18) fixed relative to the chassis (14) and a carrier arrangement (20) in the form of a sheet of flexible material (22) suspended from the support arrangement (18) and supported to form an open-top receptacle (26) for operatively receiving raw material.

### BACKGROUND TO THE INVENTION

This invention relates to the field of raw materials handling. More particularly, the present invention relates to a lightweight material carrier or hauler, typically utilised in mining and/or construction operations for hauling raw material.

Increased focus on energy efficiency and environmental impact, also in the mining and construction industries, calls for efficiency improvements in excavation and raw materials handling processes. To this end, focus is shifting towards smaller and more efficient haul trucks (smaller in this case facilitates implementation of more energy efficient technologies, greater autonomy and, in some cases, renewable energy).

Trays or beds of current haul trucks contribute a significant weight to the overall haul truck weight. Typically, trays are manufactured from a solid steel structure and provided with a polymer or rubber liner which provides protection against abrasion and wear. Current trays weigh up to about 16 tonnes while the liners contribute about a further 7 tonnes to the overall weight. The weight of the tray and liner therefore contributes a significant portion of the overall tare weight of the haul truck.

It is believed that a reduction in the weight of the tray could have a profound impact on the overall system weight of a haul truck, which could therefore play a vital role in reducing an environmental impact associated with raw material hauling. This may be achieved by an overall reduction in the energy expended during hauling, but furthermore, by bringing the total weight into an order more suitable for use of alternative, environmentally efficient and/or renewable energy resources.

It is accordingly an object of the invention to provide a haul carrier arrangement and a haul vehicle including such a haul carrier arrangement that will, at least partially, address the above disadvantages.

It is also an object of the invention to provide a haul carrier arrangement and a haul vehicle incorporating such a haul carrier arrangement which will be a useful alternative to existing haul carrier arrangements and vehicles.

### SUMMARY OF THE INVENTION

In accordance with a first aspect of the invention, there is provided a haul carrier arrangement, comprising:
a support arrangement, in use fixed relative to a structure of a haul vehicle;
a carrier arrangement in the form of a sheet of flexible material suspended from the support arrangement and supported to form an open-top receptacle for operatively receiving raw material.

The sheet of flexible material may be made up of a plurality of edge-joined sheet portions.

The flexible material may comprise a mesh or net material with a mesh or net structure such as plain weave, twill weave, Dutch weave, plain Dutch weave, twill Dutch weave, reverse Dutch weave, multiplex weave, rhomboid shape, or ring shape.

The mesh material may be a synthetic and/or a reinforced mesh. The mesh material may be reinforced by means of steel rope interweaved into the mesh. The steel rope may typically have a nominal diameter between 15 mm and 30 mm.

The flexible material may furthermore comprise externally extending reinforcements in the form of steel rope, steel wire, synthetic rope, steel strapping or synthetic strapping, which may extend peripherally and/or radially.

The carrier arrangement may be formed into a substantially bowl-shaped receptacle suspended from the support arrangement. Sides of the receptacle may converge towards a bottom portion thereof, where a material separation and/or impact absorbing arrangement may be provided. The bottom portion may comprise a grid.

At least in parts, outer surfaces of the carrier arrangement may be spaced from the support arrangement and/or other structures associated with the haul vehicle, to configure the carrier in a substantially free-hanging configuration.

The support arrangement may be fixed to, or fixed close to, a reinforced brim of the carrier arrangement.

The support arrangement may take the form of a plurality of elongate structural members extending in a substantially upwards direction from a base, for example, the members may extend obliquely relative to the base. The elongate structural members may comprise steel beams or posts. A reinforcement arrangement may extend between upper parts of neighbouring structural members. The reinforcement arrangement may extend around a brim or periphery of the carrier arrangement and may be configured as a tensioning hoop, made up of one or more tensioning ropes, rods or beam members.

Impact bumpers or pads may be provided between structural parts of the support structure and the carrier arrangement.

The carrier arrangement may include at least one, but typically more than one intrinsic sensing device. The intrinsic sensing device may comprise a sensing strand interwoven into the sheet, such as an electrical or fibre optic sensing strands, or a clamp-on sensing device, such as a radio frequency device, or more particularly, a surface acoustic wave (SAW) sensor.

The intrinsic sensing device may be configured for measuring capacitance or resistance of the raw material using electric capacitance tomography (ECT) or electrical resistance tomography (ERT).

In accordance with a second aspect of the invention there is provided a haul vehicle comprising:
a chassis associated with one of wheels and tracks;
a haul carrier arrangement according to the first aspect of the invention, wherein a support arrangement of the haul carrier arrangement is fixed to and supported by the chassis.

The haul vehicle may be specifically configured as a self-propelled vehicle, in which at least some of the wheels or tracks are driven, a trailer, a cart or a wagon.

Again, impact bumpers or pads may be provided between the chassis and the carrier arrangement of the haul carrier arrangement.

The haul vehicle may be configured as a tipping vehicle. The haul carrier arrangement may be pivotably supported relative to the chassis.

The haul carrier vehicle may include at least one, but typically a number of extrinsic sensor devices, which may be provided for operatively conducting a visual or spectroscopic evaluation of raw material carried by the haul carrier arrangement.

The one or more extrinsic sensor device may be configured for providing a visual classification of the raw material, a grading of the raw material, an estimate of particle size distribution of the raw material, an indication of a presence of particle oversize or an indication of the presence of foreign objects which may be problematic for downstream operations.

The at least one extrinsic sensor device may utilise at least one of:
X-ray fluorescence (XRF) to determine elemental composition of the raw material;
laser induced breakdown spectroscopy (LIBS) to determine an elemental composition of the raw material;
hyperspectral sensing to determine a mineralogical composition of the raw material;
visual sensing to determine lithological composition and particle size distribution (PSD) of the raw material;
nuclear magnetic resonance (NMR) to determine mineralogical composition on a volume basis;
laser induced fluorescence to determine a composition of the raw material;
pulsed fast thermal neutron activation (PFTNA) to determine elemental composition on a volume basis; and
prompt gamma neutron activation analysis (PGNAA) to determine elemental composition on a volume basis.

The haul vehicle may furthermore include an intrinsic or extrinsic detecting device configured for identifying or detecting undesirable discarded metal (tramp metal) or excavation components within raw material carried by the haul carrier arrangement.

### BRIEF DESCRIPTION OF THE DRAWINGS

The invention will now be described in more detail, by way of example only, with reference to the accompanying drawings in which:

FIG. 1 shows a schematic perspective view of a haul vehicle including a haul carrier arrangement in accordance with the invention;

FIG. 2 shows a top view of the vehicle of FIG. 1; and

FIG. 3 shows a side view of the vehicle of FIG. 1.

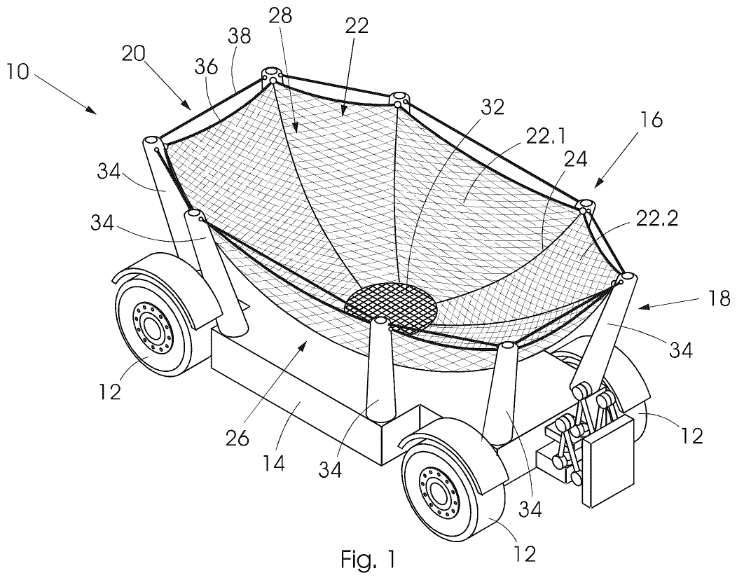

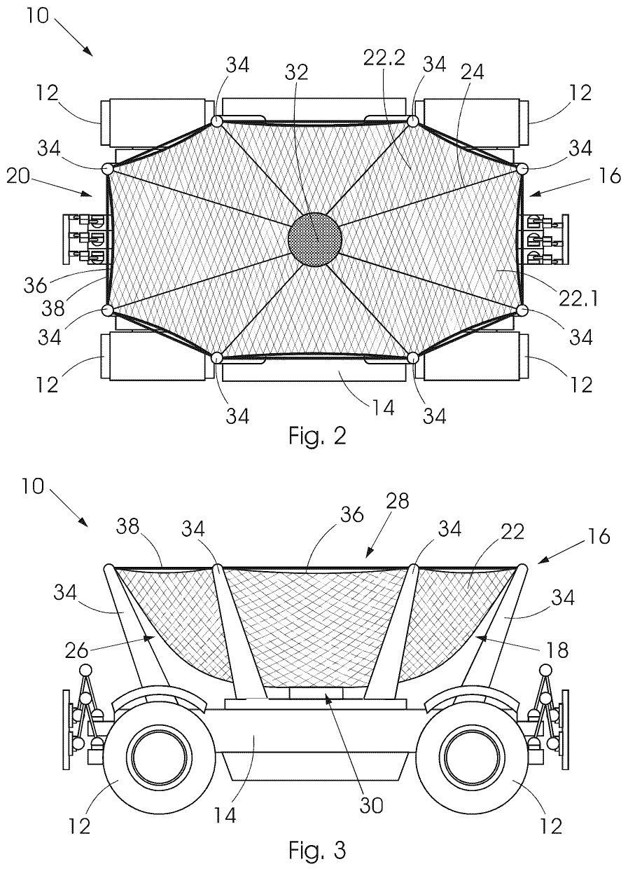

### DESCRIPTION OF THE ILLUSTRATED EMBODIMENTS

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted”, “connected”, “engaged” and variations thereof are used broadly and encompass both direct and indirect mountings, connections, supports, and couplings and are thus intended to include direct connections between two members without any other members interposed therebetween and indirect connections between members in which one or more other members are interposed therebetween. Further, “connected” and “engaged” are not restricted to physical or mechanical connections or couplings. Additionally, the words “lower”, “upper”, “upward”, “down” and “downward” designate directions in the drawings to which reference is made. The terminology includes the words specifically mentioned above, derivatives thereof, and words or similar import. It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Referring to the drawings, in which like numerals indicate like features, a non-limiting example of a haul vehicle in accordance with the invention is generally indicated by reference numeral 10. The haul vehicle may take various forms, such as a self-propelled vehicle like a haul truck, or a drawn vehicle like a trailer, cart or a wagon. The vehicle 10 is therefore not intended to be limited by its specific configuration. In some cases, for example, the vehicle 10 may be fitted with wheels 12, whereas in others, the vehicle 10 may have tracks. Also, in some embodiments, the vehicle may be configured to ride on train tracks. The vehicle 10 shown in the figures comprises a trailer.

The vehicle 10 has a chassis 14 which serves as a base for supporting payload, as is discussed more fully below.

The vehicle 10 includes a haul carrier arrangement 16 and as a result, the vehicle is configured as a haul carrying vehicle. It will be appreciated that the haul carrier arrangement 16 may form a standalone aspect of the present invention.

The haul carrier arrangement 16 comprises a support arrangement 18 which is fixed to and supported by the chassis 14.

Furthermore, the haul carrier arrangement 16 also includes a carrier arrangement 20, which is manufactured from a flexible material. The carrier arrangement 20 takes the form of an expanse or sheet 22 of flexible material and, as shown in the embodiment shown in the figures, may typically be made up of a number of sheets (reference numerals indicating separate sheets have suffixes) of flexible material which are joined along adjoining edges 24 into the single expanse or sheet 22. Typically, the separate sheets are non-rectangular, and rather, are substantially triangular or trapezoidal in shape, such that the sheet 22, when unladen, defines a substantially bowl-shaped receptacle 26 with an open top end 28. The sheet may converge towards a bottom portion 30 of the receptacle 26. The bottom portion 30 of the receptacle 26 is formed by a grid 32, which is a replaceable or consumable protective element provided in an area of high impact, load and wear during loading.

The sheet 22 is suspended from the support arrangement 18 such that the receptacle 26 hangs from the support arrangement 18 towards the chassis 14. The support arrangement 18 therefore supports the sheet 22 in such a way that the top end 28 is kept open and that that the receptacle 26 is formed. In use, the receptacle 26 receives a load of raw material (not shown) to be hauled by the vehicle 10 through the open top end 28.

The sheet 22 is manufactured from a mesh material, and particularly, from a synthetic mesh material. Typically, the mesh may be a high-tensile steel wire or rope mesh, having a rhomboid or ring mesh shape. The sheet 22 may take the form of a net. Typically, the mesh or net may be made up of rope or wire having diameters in the range of between 2 mm and 6 mm, although the use of larger diameter rope or wire is permissible. Each rope or wire may have a typical tensile resistance of between about 20 kN and 40 kN and may be coated with a corrosion protective coating. In some examples, the mesh may be of the kind known as GEOBRUGG® High-tensile steel wire net ROCCO® 7/3/300 12/3/300 16/3/300 19/3/300, GEOBRUGG® High-tensile spiral rope net SPIDER® S 4-130 or GEOBRUGG® High-tensile steel wire mesh MINAX® 80/4.6.

Furthermore, the mesh may be a reinforced mesh, which is reinforced by means of a steel rope or wire which is interweaved into the mesh. The steel rope or wire may in some cases be of a synthetic or composite construction. The steel rope or wire may typically have a nominal diameter between 15 mm and 30 mm and may have a minimum breaking load of between 250 kN and 400 kN. The rope may include a corrosion protective coating, such as galvanising. The rope may be arranged in a grid, with spacing between adjacent ropes between 0.2 m and 1 m.

Apart from the interweaved steel rope or wire, the carrier arrangement may also comprise further reinforcement, such as externally extending (circumferentially, peripherally and or radially extending) reinforcements in the form of steel rope or wire, synthetic rope or strapping or the like. Such interweaved and/or external reinforcements are provided to add structural integrity to the sheet, to limit a permissible amount of stretching and/or elastic deformation of the sheet and to improve the sheets'load and abrasion handling capabilities.

The mesh material may have a mesh structure such as plain weave, twill weave, Dutch weave, plain Dutch weave, twill Dutch weave, reverse Dutch weave, or a multiplex weave.

The mesh material typically has a mesh size dictated by type of raw material or commodity carried by the vehicle 10 and by the type of down-stream process. The relevance of the aperture size of the mesh is discussed more fully below.

The support arrangement 18 is made up of a number of elongate steel structural members, beams or posts 34 which are fitted to, and extend from, the chassis 14. The posts 34 project in a substantially upward direction, albeit obliquely relative to the chassis 14. The posts 34 flare upwards and outwards relative to the chassis 14.

A brim 36 of the sheet 22 is fixed to upper portions of the posts 34. The brim itself may typically be reinforced. Furthermore, a tensioning hoop 38 is provided around the upper portions of the posts 34 to provide structural rigidity and support to the support arrangement 18. The tensioning hoop 38 takes the form of a steel rope or cable, or steel rods, tubes or beams, which extend between the posts 34. The hoop 38 may be manufactured from a single piece or may comprise separate interconnected pieces.

The configuration may typically be such that the sheet 22 is free-hanging, and therefore does not directly urge against the posts 34 or chassis (with the exception of the bottom portion 30). Sides of the sheet 22 may therefore be largely unsupported and dynamic displacement of parts of the sheet 22 relative to the support arrangement 18 and chassis 14 is therefore possible.

That said, impact bumpers or pads (not shown) may be provided over sheet-facing surfaces of the posts 34 and/or chassis 14, to absorb impacts and limit abrasion and wear caused during loading of the raw material into the receptacle 26.

It is believed that the dynamic displacement of the sheet 22 relative to the chassis 14 may assist in absorbing impacts and loads caused during loading.

Since the sheet 22 is manufactured from a mesh material a basic separation function of the raw material may be performed, in that material fragments smaller than the aperture size of the mesh (and of the grid 32) may be allowed to fall through the apertures of the mesh. It is believed that the free-hanging arrangement of the receptacle 26 which allows displacement of the sheet relative to the support arrangement 18 may aid in the separation process in that smaller particles and fragments may be urged towards the sheet and through the apertures. Therefore, by the time the raw materials are deposited to a secondary process (after having been hauled by the vehicle 10), some smaller and unusable fragments and particles may already have been removed or expelled from the raw material and therefore relieves some of the separation and/or sorting burden on downstream processes.

Provision is furthermore made for the sheet to be made up of more than one sheet, arranged in cascading fashion. The different sheets may be associated with different aperture sizes, to facilitate sorting of materials based on different nominal fragment sizes.

It will be appreciated that vibrations transferred from the chassis may assist in the sorting function associated with the sheet 22. Additionally, vibration may externally be induced by means of forced vibration (by way of a shaker) exerted on the sheet 22, to facilitate further or more effective sorting.

In some examples, the carrier arrangement 20 includes at least one, but typically a number of intrinsic sensing devices (not shown), which may be used to sense various parameters associated with the raw material carried, deflections, loads, shocks and vibrations transferred to the sheet 22, abrasion and wear of the sheet 22, and the like. For example, measuring of dynamic strain signals from the intrinsic sensing devices during loading or transport of the raw material may enable determination of particle size distribution or the identity of particle oversize on a binary basis.

It is believed that the present configuration of the carrier arrangement 20 may furthermore lend itself to the use of live intrinsic and interwoven sensing devices measuring capacitance or resistance of the raw material using electric capacitance tomography (ECT) and/or electrical resistance tomography (ERT).

The intrinsic sensing devices may therefore provide an indication (in real-time, intermittently or historically) of a condition of the carrier arrangement 20 or even the vehicle 10.

The sensing devices may take the form of sensing strands interwoven into the sheet 22. Typically, the sensing strands may take the form of electrical or fibre optic sensing strands. Alternatively, the sensing devices may take the form of clamp-on sensing devices utilising radio frequency device or surface acoustic waves to measure the parameters (here, typically limited to mechanical measurements).

Furthermore, the vehicle 10 may have extrinsic sensing capabilities made possible by the visual availability of the raw material, facilitated by the substantially transparent sheet 22 (in cases where a mesh or net is used). The extrinsic sensing capabilities are provided by visual or spectroscopic devices which conduct a visual or spectroscopic evaluation of the raw material. The extrinsic sensing capabilities are therefore focussed on an evaluation of the raw material, and may, for example, be used to classify or grade the material.

Further, machine vision methods may exploit visibility through the mesh or net to estimate the particle size distribution, monitor for the presence of particle oversize and/or identify foreign objects which may be problematic for downstream operations.

Either intrinsic or extrinsic metal-detecting strands may be introduced to identify the undesirable presence of discarded steel (so-called “tramp metal”), such as bucket teeth or steel in stockpiles from previous mining operations.

Because of the substantially transparent sheet 22, it is believed that at least some of the below sensing techniques and outcomes may be enabled:
X-ray fluorescence (XRF): elemental composition of the raw material;
laser induced breakdown spectroscopy (LIBS): elemental composition of the raw material;
hyperspectral: mineralogical composition;
visual: lithological composition, particle size distribution (PSD);
nuclear magnetic resonance (NMR): mineralogical composition on a volume basis;
laser induced fluorescence: composition; and
nuclear methods such as pulsed fast thermal neutron activation (PFTNA) or prompt gamma neutron activation analysis (PGNAA): elemental composition on a volume basis.

It will be appreciated that both the intrinsic and extrinsic sensing may be conducted at predetermined discrete time intervals to provide improved representation over isolated measurements. The introduction of vibration energy between measurements may facilitate net transport of granules within the mesh.

It will furthermore be appreciated that sensing may occur during loading or transportation of the raw material.

The use of a sheet 22 instead of a solid bin or tray with an associated liner results in a significant reduction of the overall weight of the vehicle 10, in turn resulting in a more efficient (energy efficient and therefore environmentally friendly) hauling process.

Furthermore, it is believed that the sheet is relatively easy, quick and cost efficient to replace (especially when compared to solid bins or even synthetic bin liners) should same become worn out.

Also, the substantially transparent sheet 22 is believed to be provide unique benefits from an intrinsic and extrinsic sensing point of view.

It will be appreciated that the above description only provides an example embodiment of the invention and that there may be many variations without departing from the spirit and/or the scope of the invention.

For example, some of the abovementioned functions may be obtainable with a sheet manufactured from a solid material (rather than a mesh) and such a sheet therefore forms part of the present invention.

Furthermore, the support arrangement may take various other forms and need not comprise separate posts extending from the chassis. For example, the support arrangement may theoretically take the form of a frame member which extends along a periphery of the open top end 28.

It will be appreciated that the vehicle may in some examples be configured as a tipping vehicle, and that the support arrangement 18 and/or the chassis 14 and/or another structural part of the vehicle 10 may be configured to lift or tip the carrier arrangement 20 to cause raw materials carried therein to be dumped.

Furthermore, in some cases, the carrier arrangement may be detachably supported by the support arrangement 18. In such cases, the carrier arrangement 20 (formed into a bag-like configuration) may be detached and removed from the vehicle 10 (such as by crane) to allow disposal of the material carried therein or to allow for further transportation (separate from the vehicle) or even storage of the raw material.

It will easily be understood from the present application that the particular features of the present invention, as generally described and illustrated in the figures, can be arranged and designed according to a wide variety of different configurations. In this way, the description of the present invention and the related figures are not provided to limit the scope of the invention but simply represent selected embodiments.

The skilled person will understand that the technical characteristics of a given embodiment can in fact be combined with characteristics of another embodiment, unless otherwise expressed or it is evident that these characteristics are incompatible. Also, the technical characteristics described in a given embodiment can be isolated from the other characteristics of this embodiment unless otherwise expressed.

1-32. (canceled)

33. A haul carrier arrangement, comprising:
a support arrangement, in use fixed relative to a structure of a haul vehicle;
a carrier arrangement in the form of a sheet of flexible material suspended from the support arrangement and supported to form an open-top receptacle for operatively receiving raw material.

34. The haul carrier arrangement according claim 33, wherein the flexible material comprises a mesh or net material; wherein the mesh material comprises at least one of: a synthetic mesh; and a reinforced mesh; and wherein the mesh or net material has a mesh or net structure selected from a list comprising: plain weave, twill weave, Dutch weave, plain Dutch weave, twill Dutch weave, reverse Dutch weave, multiplex weave, rhomboid shape, and ring shape.

35. The haul carrier arrangement according to claim 34, wherein the mesh material is reinforced by means of steel rope interweaved into the mesh, and wherein the steel rope has a nominal diameter between 15 mm and 30 mm.

36. The haul carrier arrangement according to claim 34, comprising externally extending reinforcements extending at least one of peripherally and radially and wherein the externally extending reinforcements take the form of one of: steel rope; steel wire; synthetic rope; steel strapping and synthetic strapping.

37. The haul carrier arrangement according to claim 33, wherein the carrier arrangement is formed into a substantially bowl-shaped receptacle suspended from the support arrangement and wherein sides of the receptacle converge towards a bottom portion thereof, and wherein the bottom portion comprises a material separation arrangement.

38. The haul carrier according to claim 37, wherein the bottom portion comprises a grid.

39. The haul carrier arrangement according to claim 33, wherein at least in parts, outer surfaces of the carrier arrangement are spaced from the support arrangement and/or other structures associated with the haul vehicle, to configure the carrier in a substantially free-hanging configuration.

40. The haul carrier arrangement according to claim 33, wherein the support arrangement is fixed to, or fixed close to, a brim of the carrier arrangement and wherein the brim is reinforced.

41. The haul carrier arrangement according to claim 33, wherein the support arrangement comprises a plurality of elongate structural members in the form of steel beams or posts extending from or relative to a base in one of: a substantially upwards direction; and obliquely relative to the base.

42. The haul carrier arrangement according to claim 41, wherein a reinforcement arrangement extends between upper parts of neighbouring structural members, wherein the reinforcement arrangement extends around a brim or periphery of the carrier arrangement and is configured as a tensioning hoop and wherein the reinforcement arrangement comprises one of: one or more tensioning ropes; one or more tensioning rods; and one or more beam members.

43. The haul carrier arrangement according to claim 33, comprising impact bumpers or pads between structural parts of the support structure and the carrier arrangement.

44. The haul carrier according to claim 33, wherein the carrier arrangement includes at least one intrinsic sensing device, comprising at least one of i) a sensing strand interwoven into the sheet, selected from electrical and fibre optic sensing strands; and ii) a clamp-on sensing device in the form of a radio frequency device, including a surface acoustic wave sensor.

45. The haul carrier according to claim 44, wherein the intrinsic sensing device is configured for measuring capacitance or resistance of the raw material using electric capacitance tomography (ECT) or electrical resistance tomography (ERT).

46. A haul vehicle comprising:
a chassis associated with one of wheels and tracks;
a haul carrier arrangement according to claim 33, wherein a support arrangement of the haul carrier arrangement is fixed to and supported by the chassis.

47. The haul vehicle according to claim 46, configured as at least one of: a tipping vehicle, wherein the haul carrier arrangement is pivotably supported relative to the chassis; a self-propelled vehicle, in which at least some of the wheels or tracks are driven; a trailer; a cart; and a wagon.

48. The haul vehicle according to claim 46, comprising impact bumpers or pads between the chassis and a carrier arrangement of the haul carrier arrangement.

49. The haul vehicle according to claim 46, including at least one extrinsic sensor device provided for operatively conducting one of a visual and spectroscopic evaluation of raw material carried by the haul carrier arrangement.

50. The haul vehicle according to claim 49, wherein the at least one extrinsic sensor device is configured for providing one of: a visual classification of the raw material; a grading of the raw material; an estimate of particle size distribution; an indication of a presence of particle oversize; and an indication of the presence of foreign objects which may be problematic for downstream operations.

51. The haul vehicle according to claim 49, wherein the at least one extrinsic sensor device utilises at least one of:
X-ray fluorescence (XRF) to determine an elemental composition of the raw material;
laser induced breakdown spectroscopy (LIBS) to determine an elemental composition of the raw material;
hyperspectral sensing to determine a mineralogical composition of the raw material;
visual sensing to determine lithological composition and particle size distribution (PSD) of the raw material;
nuclear magnetic resonance (NMR) to determine mineralogical composition on a volume basis;
laser induced fluorescence to determine a composition of the raw material;
pulsed fast thermal neutron activation (PFTNA) to determine elemental composition on a volume basis; and
prompt gamma neutron activation analysis (PGNAA) to determine elemental composition on a volume basis.

52. The haul vehicle according to claim 46, including one of an intrinsic or extrinsic detecting device configured for identifying or detecting undesirable discarded metal or excavation components within raw material carried by the haul carrier arrangement.
